(19) United States
(12) Patent Application Publication
(10) Pub. No.: US 2016/0151211 A1
(43) Pub. Date: Jun. 2, 2016

(54) Garment

(71) Applicant: THE UNIVERSITY OF DUNDEE, Dundee (GB)

(72) Inventor: John Biddlestone, Falkirk (GB)

(21) Appl. No.: 14/907,481

(22) PCT Filed: Jul. 24, 2014
(86) PCT No.: PCT/GB2014/052271

§ 371 Date: Jan. 25, 2016

(30) Foreign Application Priority Data
Jul. 24, 2013 (GB) 1313199.0

(51) Int. Cl. A61F 13/14; A61F 13/00; A41C 3/00

(52) U.S. Cl. CPC A61F 13/145; A41C 3/0064; A41C 3/0092; A41C 3/0035; A61F 13/00063

(57) ABSTRACT

The present invention relates to a garment that will provide support and/or expedite the healing of an injured or wounded breast and/or be used to re-symmetrise (surgically) wounded and/or asymmetrical breasts. The garment may also be used to simulate the natural appearance of a breast or breasts following surgery or trauma resulting in the loss of mammary tissue.

### FIELD OF THE INVENTION

The present invention relates to a garment for providing support to an injured or wounded breast.

### BACKGROUND OF THE INVENTION

A large number of women undergo reconstructive breast surgery every year. Some of these women undergo such treatment for cosmetic reasons, e.g. breast reductions, breast enlargements and breast symmetrisation procedures. However, many of those who undergo reconstructive breast surgery have been diagnosed with breast cancer. Treatment of breast cancer often involves some form of surgery, e.g. a mastectomy or lumpectomy. Following this, a significant number of these patients will undergo reconstructive breast surgery to restore a natural appearance or symmetry to the breasts. Women who undergo reconstructive breast surgery will often require more than one operation (commonly between 3 and 4 operations) to achieve symmetry between their new breasts.

There are many problems associated with the post-operative care of the breast such as the risk of infection, wound cosmesis, monitoring of tissue viability and the pain experienced by a patient in the post-operative period.

### SUMMARY OF THE INVENTION

The present invention relates to a garment that will provide support and/or expedite the healing of an injured or wounded breast and/or be used to re-symmetrise (surgically) wounded and/or asymmetrical breasts. The garment may also be used to simulate the natural appearance of a breast or breasts following surgery or trauma resulting in the loss of mammary tissue.

Thus, according to a first aspect of the invention there is provided a garment, for example a bra, for providing support to a breast, said bra comprising a wound dressing material.

It should be noted that throughout this specification the term comprising is used to denote that embodiments of the invention “comprise” the noted features and as such, may also include other features. However, in the context of this invention, the term “comprising” encompasses embodiments in which the invention “consists essentially of” the relevant features or “consists of” the relevant features. For example, while this invention provides a bra comprising a wound dressing material, the invention may further provides bras consisting essentially of wound dressing material and bras consisting of wound dressing material.

The bra of this invention comprises at least one cup—the cup being formed and adapted to at least partially envelope and/or support a breast. The bra may comprise two cups. A strap extending from at least one cup is configured to mount the bra to the wearer. The strap may be configured to be worn around an individual's back and ribcage. The strap may be configured to be releasably connectable to the cup at one or both ends. The bra may comprise one or more shoulder straps.

In use, the bra may be worn by a patient to provide support to a wounded breast or breasts. A breast wound may occur following trauma, for example an accident or following a surgical procedure—as such, the term “wound” may encompass an injury and/or surgical wound. A bra of this invention may be used to support one or both breast(s) in a post-operative period. The bra of this invention may be worn by a patient following surgery to remove all or part of a breast or all or part of the mammary tissue. The bra disclosed herein may find particular application in the provision of support to a breast or breasts following, for example, mastectomy, lumpectomy and/or reconstructive procedures.

The bra may comprise or be formed of, at least in part, a wound dressing material. The wound dressing material may comprise, for example, any material suitable for direct application to a wound. The wound dressing material may be a medical material, a therapeutic material and/or a wound protecting material.

The wound dressing material may comprise an active agent. For example, the wound dressing material may comprise an antimicrobial agent. One of skill will appreciate that antimicrobial agents for use in this invention may exhibit an ability to inactivate a microorganism, inhibit the growth of a microorganism and/or kill a microorganism. The wound dressing material may comprise an antibiotic. The wound dressing material may comprise an anti-bacterial agent, an anti-fungal agent and/or an anti-viral agent. The wound dressing material may comprise or further comprise an antimicrobial metal, an antibacterial metal and/or a material/metal having an oligodynamic effect. The wound dressing material may comprise silver.

The wound dressing material may comprise or further comprise an active agent which promotes, enhances and/or facilitates wound healing and/or has a therapeutic effect. To this end the wound dressing material may comprise or further comprise an active agent and/or structural properties and/or a composition that promotes, facilitates and/or affects wound healing and/or has a therapeutic effect. For example, the wound dressing material may comprise proteins, peptides, amino acids, antibodies (and/or antigen binding fragments thereof), immunomodulatory compounds (for example a cytokine), metal oxides (for example, zinc oxide and the like), clotting factors, anti-inflammatory agents, chemotherapeutic agents, sterilizing agents and the like.

An active agent may be applied to, coated on or impregnated into the wound dressing material. Additionally or alternatively, an active agent may be incorporated into the wound dressing material; for example, a thread, yarn, wire or fabric comprising or formed of an active agent or, for example, an oligodynamic metal, for example silver (and/or other oligodynamic material), may be woven, incorporated and/or stitched into or with the wound dressing material. Optionally, an active agent may be formulated for delayed and/or controlled release once coated, impregnated or incorporated on, in or into a wound dressing material of the bra. For example, an active agent may be encapsulated within a degradable capsule or coating. For example, a degradable capsule may comprise a lipid or wax containing vesicle.

Accordingly, the invention provides a bra for providing support to a breast, said bra comprising a wound dressing material, wherein the wound dressing material comprises one or more active agents. The one or more active agents may comprise, for example an antimicrobial agent and/or agent(s) which promote, facilitate or affect wound healing. The wound dressing material may comprise an antibiotic and/or oligodynamic material.

One of skill will appreciate that a bra comprising wound dressing material which itself comprises either an antimicrobial agent and/or agents which promote, facilitate and/or affect wound healing may, in use, increase the rate of wound healing and/or reduce the risk of wound infections—particularly infections of wounds and surgical wounds as might occur in a post operative period. For example, use of the bra provided by this invention may increase the rate at which surgical wounds heal and/or reduce the risk of wound infections caused or contributed to by bacteria, fungi and/or viruses.

The wound dressing material may comprise or further comprise a wicking and/or absorbent material. Materials of this type may assist the drainage of bodily fluids, such as pus, lymph or the like, from a wound.

The wound dressing material may comprise a breathable material. The breathable material may comprise a gauze-like material or a mesh-like material. In such embodiments, the wound dressing material may allow circulation of air to the wound to promote wound healing.

Representative examples of wound dressing materials for use in this invention include, but are not limited to, antibacterial jersey fleece fabric (obtained from Lee Yaw Textile Co. Ltd), Torex® Makspec® (obtained from Toray), Infect-Protect (obtained from Raumedic®) and/or antimicrobial protected fabrics (obtained from Fab Industries). Any one of these materials may be modified to comprise an active agent of the type described above.

The wound dressing material may comprise a gauze pad.

As used herein the term ‘gauze pad’ may refer to a pad comprising synthetic absorbent materials and/or naturally occurring absorbent materials. Representative examples of absorbent materials for use in this invention include, but are not limited to, fabrics comprising cotton, bamboo, Oasis Super Absorbent Fibers (Oasis SAF®) and/or sodium polyacrylate.

Additionally, or alternatively, the bra may provide increased wound cosmesis and/or may reduce pain experienced by a patient with a wounded breast or in a post-operative period.

The bra may comprise one or more additional materials. The one or more additional materials may be dissimilar to the wound dressing material. The bra may comprise non-wound dressing and/or non-medical materials. The bra may comprise an additional material selected from cotton, nylon, Lycra, lace, elastane, and/or mixtures of such materials. The one or more additional materials may improve an aesthetic appearance of the bra and/or may improve structural properties of the bra. For example, the one or more additional materials may improve the strength and/or elasticity of the bra.

The bra may comprise an indicator material configured to indicate to an individual when a wound dressing material should be removed and/or replaced e.g. the indicator may indicate when a wound dressing material is fully saturated or when a wound has become infected. Alternatively, or additionally, the indicator may indicate when an active agent is no longer effective, has expired, “gone off” and/or has been consumed. The material may comprise an indicator, such as a pH indicator, which changes color upon contact with bodily fluids and so indicates when the wound dressing material is fully saturated or when a wound is infected.

The bra or a cup thereof may comprise layers of material, for example, the bra, at least in certain areas, such as in a cup, may comprise a layered structure. The bra may comprise layers of wound dressing materials and/or of additional materials, e.g. non-medical materials.

The layers may be attached, releasably or permanently to one another. For example, the layers may be permanently attached to one another by an adhesive or by a stitched seam or may be attached by a fastening mechanism, such as a hook and loop type mechanism e.g. Velcro®, buttons, press studs, zip fasteners, or the like.

The bra or a cup thereof may comprise a laminate material. For example, two or more layers of material may be laminated together.

The bra or a cup thereof may comprise an inner layer of material and an outer layer of material. The bra may also comprise an intermediate layer of material, or a plurality of intermediate layers. At least one of the inner, outer and/or intermediate layers may comprise a wound dressing material. Other layers may comprise a wound dressing material and/or another (non-wound dressing) material as described above.

As used herein, the inner layer may be defined as a layer which is adjacent to and/or contacts the skin of an individual. As used herein, the outer layer of the bra or a cup thereof may be defined as a surface layer on an opposing face to the inner layer which faces outwardly away from an individual. As used herein, an intermediate layer may be defined as a layer which is disposed between the inner and outer layers of the bra.

The inner layer may comprise a wound dressing material. The outer layer may comprise a non-wound dressing material, such as a non-medical material and/or an aesthetically pleasing material. In some embodiments, the outer layer and/or an intermediate layer may comprise a liquid impermeable material, such as a waterproof material, which may reduce the amount of water contacting the wound or may assist in maintaining a dry environment for a wound. Additionally or alternatively, such a construction may mitigate the risk of seepage of wound fluid onto an individual's clothing. For instance, the bra may comprise a gauze pad laminated to a liquid impermeable plastic material.

One or both cups may comprise the wound dressing material. For instance, in cases where only one breast has a wound and/or has been operated upon, e.g. in a unilateral breast reconstruction procedure, the cup contacting or supporting the treated breast, e.g. the reconstructed breast, may comprise the wound dressing material and the cup contacting or supporting the untreated breast may be formed of an alternative material e.g. cotton, Lycra, nylon, elastane, lace or the like. For instance, the cup contacting or supporting the untreated breast may comprise any material typically found in a conventional bra.

The wound dressing material of the garments of this invention may be confined to certain areas, for example certain areas, regions or zones of one or both cups of a bra disclosed herein. The positioning of the wound dressing material may correspond to known surgical incision/wound patterns. Those areas, regions or zones of the bra that combine wound dressing material may be further provided with flaps or covers movable between first (open) and second (closed) positions. The flaps or covers may be fixed to the garments of this invention and releasably securable in the second (closed) position. In the second or closed position, the flap or cover substantially 9 or at least partially) covers the region, area or zone of wound dressing material. The flap or cover may define a pocket for receiving further wound dressing material and/or a prosthesis.

The bra may define a window. The window may allow the user and/or a third party to monitor the healing progress of a wounded breast. The window may allow the healing progress to be monitored directly, by allowing a third party to observe at least part of a wound. Alternatively, or additionally, the window may allow the healing process to be monitored indirectly, e.g. by exposing an area of the breast, for example an area of the breast proximal to and/or associated with, a wound. Additionally, or alternatively, the window may expose and/or allow a user and/or third party to observe, all or part of the wound dressing material.

The bra may define a plurality of windows, each exposing all or part of a breast wound, area of the breast associated with or proximal to, a wound and/or wound dressing material of the bra.

A window may comprise an aperture defined by the bra. The window may comprise a transparent, opaque and/or mesh-like material disposed within the aperture defined by the bra. The window may not allow direct observation of the wound but may comprise (i.e. have disposed within) a wound dressing material.

A window may be defined by a cup of the bra. The window defined by the bra may expose an area of the breast for observation. Additionally or alternatively, a window defined by the bra may expose a wound so that it may be directly contacted to or with a wound dressing material. The wound dressing material may then be regularly observed so that it can be replaced as required.

The window may comprise a flap or cover configured to cover or obscure the aperture and/or the window may comprise a flapped or covered window. The flap or cover (referred to hereinafter as “flap”) may be moveable between a closed position and an open position. In the closed position, the flap may cover the aperture. In an open position, the flap may be positioned away from the aperture. The flap may comprise a transparent, opaque and/or liquid impermeable material. The flap may protect a wound, breast tissue in the vicinity of, and/or associated with, a wound or wound dressing material of a bra or window thereof from liquid ingress. The flap may assist in maintaining a dry environment surrounding a wound. Observation through the window may be possible when the flap is in the open position. Alternatively, observation through the window may be possible when the flap is both in the closed and open positions, for example, when the flap comprises an opaque and/or transparent material or the like. The bra may comprise a plurality of flapped windows. The flap may define a pocket into which a reconstructing or re-symmetrising prosthesis may be inserted. The flap may also be used to trap or secure a piece of wound dressing material between the flap and the bra cup or any window thereof.

The (or each) window defined by the bra may be configured for location over a certain area of the breast, e.g. over a wound, a nipple and/or areole area or proximal to any of these sites, to allow a medical professional, such as a nurse or doctor, to monitor the healing progress of a wounded breast, e.g. to monitor tissue health, viability and healing, in a post-operative period. Such an observation window may allow a medical professional to monitor the healing progress without the need for removing either the bra and/or any wound dressing material thereof. A bra of this invention may define one or more windows in pre-determined positions, which positions are intended to correspond to areas of the breast affected by surgical procedures.

As such, this invention provides a bra for providing support to a breast, said bra comprising a wound dressing material and defining one or more windows, each window independently exposing a wound, an area of the breast and/or wound dressing material for observation. The wound dressing material may comprise an active agent.

The bra may comprise and/or define a pocket. One or both cups may define a pocket. The pocket may define an opening through which the wound dressing material (for example a disposable dressing pad) and/or a prosthesis may be inserted for location within the pocket.

The opening (a pocket opening) may be concealed and/or located or positioned such that when worn, the opening is not accessible and/or is closed. For example, the opening may be defined by an inside and/or an outside surface of the bra (an inside surface being a surface which, when the bra of this invention is worn, is in contact with or faces the wearer). An opening may be defined by an inside and/or outside surface of one or both cups of the bra. The opening may be defined by a lateral component of the bra. A lateral component being a component of the bra which, when worn, spans the side part (the part between the anterior and posterior planes) of the wearer's body. For example, a pocket may be defined by those parts of the bra which comprise a cup component and the opening through which the pocket may be accessed may be defined by a lateral component of the bra. In use (i.e. when worn) a pocket opening defined by a lateral component of a bra may be closed through compression, for example compression of the opening against the wearer. With such an arrangement, access to the pocket defined by the bra through an opening defined by a lateral component of the bra will be restricted when the bra is correctly worn and permitted when the bra is not worn or when there is no compression on the lateral bra component—for example when the bra is worn using only the shoulder straps and not, for example, the chest band (the part of the bra that runs below the breast and around the back).

A prosthesis (e.g. for insertion into a pocket defined by a bra of this invention) may comprise a variety of shapes and/or may be configured to provide a flattering profile to the wearer. The prosthesis may be configured to restore a pre-surgery profile to a breast and/or to provide a symmetrised breast profile to an individual. For example, the prosthesis may comprise a rounded or teardrop-like surface profile. The prosthesis may comprise gel, silicon, water and/or saline. The prosthesis may be a re-symmetrising or a re-constructing prosthesis.

The bra (or each cup thereof) may comprise a plurality of wound dressing material pieces—each piece being independently removably attachable to the bra. The bra (or each cup thereof) may comprise, define or be divided into different zones. Each cup may independently comprise, define or be divided into different zones. Each zone may be defined by a pocket. The interior of each pocket may be accessible by an opening also defined by the bra—such openings (and the associated pockets) are defined above. Each zone may independently comprise or be adapted to receive, a piece of wound dressing material. For example, each zone may independently comprise or be adapted to receive a removably attachable piece of wound dressing material. The location of the zones may correspond to known surgical wound and/or scar patterns. The wound dressing material may be removably attachable to one or more of the zones via, for example, a hook-and-loop type mechanism (such as Velcro®) or using another fastening mechanism, such as buttons, press studs, zip fasteners or the like. Different materials and/or wound dressing materials may be releasably attached to the different zones.

In use, wound dressing material may be located in or attached or fitted to the zone or zones of the bra that correspond(s) to an expected wound and/or scar pattern on a patient before the bra is fitted to and/or worn by a patient. The wound dressing material may be replaced as necessary—for example when saturated with fluid and/or as any active agent becomes ineffective.

In addition to any flap of material which can be used to cover an aperture and/or window defined by a bra of this invention, the bra may comprise one or more movable (material) flaps which are (releasably) fixed to the bra, for example a cup portion of the bra, and which can be releasably fixed thereto. The flap may comprise one or more layers of material and may comprise a wound dressing material. The flap may define a pocket and an opening thereto. The pocket may be formed and adapted to receive a wound dressing material—for example a disposable dressing pad or a prosthesis as described herein. An outer surface of the flap may comprise any type of material, including any of those described herein. An outer surface may comprise a water-impermeable, breathable material. The flap may additionally or alternatively be used to trap, for example a piece of wound dressing material and/or a prosthesis against a part of the cup. For example, as described elsewhere, the flap may, in use, cover or obscure an aperture or window defined by the bra of this invention and in such cases, the flap may be used to releasably secure or trap a disposable wound dressing pad adjacent the aperture or window such that any healing wound is kept clean and fluid (for example lymph) drained or wicked therefrom. For example, a flap of a bra of this invention may define a pocket into which a prosthesis is inserted; the flap may then be closed against a cup of the bra trapping therebetween a disposable wound dressing pad.

The bra, the wound dressing material and/or one or both of the cups may be disposable.

The bra may be asymmetric. As used herein, the term “asymmetric” may relate to a bra comprising cups of different sizes. For example, one cup may be larger or smaller than the other cup. An asymmetric bra may be configured to be worn by an individual who may have breasts of different sizes, e.g. following or as a result of surgery.

The bra may have a modular construction. For example, the bra may be formed of multiple components, e.g. a first and second component. The first component may comprise a bra cup and the second component may comprise a bra cup. The first and second components may be configured for attachment to each other so as to form a bra comprising two cups. The first and second components may be releasably connectable. The first and second components may be releasably connectable anteriorly, e.g. between the breasts of an individual via a front connector. Additionally, or alternatively, the first and second components may be releasably connectable posteriorly, e.g. at the back of an individual via a back connector.

The first and second components may be releasably connectable at a plurality of discrete positions. The front connector may be releasably connectable at a plurality of discrete positions to accommodate any variation in the intermammary space between individuals. The back connector may be releasably connectable at a plurality of discrete positions at the back of an individual, e.g. to allow for variations in the size of an individual's ribcage, e.g. between different individuals.

The front connector may comprise a central connector. One end of the central connector may be releasably connectable to a first cup and the other end of the central connector may be releasably connectable to a second cup. The central connector may be insertable into pockets defined by the cups and/or attachable to each of the first and second cups internally within the pockets. The central connector may comprise connector elements that meet with their partner connector elements within the pockets.

Either cup or both cups may comprise a tab and/or a pocket. The front connector may comprise a tab extending from the first and/or second cup and a pocket comprised on the first and/or second cup. The tab may be insertable into the pocket. The tab and pocket may comprise connector elements which may be connectable after insertion of the tab into the pocket.

The front and/or back connector may comprise connector elements to provide a releasable connection. The connector elements may comprise hooks and loops, e.g. metallic or plastic hooks and loops, Velcro®, press studs, poppers, 3M Dual Lock® and the like. The connector elements may be outwardly facing, may connect in a pocket and/or may be covered by a sleeve. Such configurations may reduce and/or prevent abrasion or irritation of an individual's skin by the connector elements.

An area comprising, surrounding, in the vicinity of and/or associated with, a connector element may be reinforced. Reinforcement may comprise increasing strength and/or rigidity of the area. The area may be reinforced by the inclusion of rigid material into the bra, treatment of a material (e.g. by heat or chemical treatment or otherwise) comprised in the bra to increase its rigidity and/or strength, and/or by stitching over the area. For example, a rigid material may be positioned and/or attached between layers of material in the bra to strengthen that area. Such reinforcement may provide a secure anchorage area for the connector element and/or may provide an improved connection between the different components of the bra.

In such a modular system, advantageously, an individual may be fitted with bra cups of different sizes to provide improved support, e.g. improved physical support, such as support against excessive movement, to both breasts in, for example, a post-operative period. For example, after breast reconstructive surgery, an individual may have asymmetric breasts but by using a bra having a modular construction, cups of different sizes can be easily and readily fitted to the individual to provide a bra with improved levels of support for both breasts.

The cups may comprise a support element configured to provide support to the underside of a breast. The support element may comprise a rigid or semi-rigid material, such as a plastics material or a metallic material. The support element may comprise underwire, such as a padded underwire to prevent irritation of a breast. The support element may comprise a substantially planar, paddle-like and/or arcuate shape. The support element may comprise a substantially arcuate shape terminating in enlarged head portions at each opposing end. The head portions may be rounded and/or flattened, which may reduce the risk of irritation or abrasion on the breast area. The support element may comprise a lateral head portion and a medial head portion. The lateral head portion may be larger than the medial head portion. Such a construction may provide an improved amount of lateral support to the breast, whilst reducing congestion in an intermammary space.

According to a second aspect of the invention there is provided a medical kit comprising a plurality of bras according to the first aspect of this invention, wherein each bra comprises one cup configured for connection to another cup. The kit comprises a plurality of first bra cups and second bra cups, wherein the first and second bra cups are configured for interchangeable attachment to one another to provide a bra comprising two cups, the bra comprising a wound dressing material.

The first cups and second cups may be different sizes. As explained, the connecting together of differently sized first and second cups provides an asymmetric bra.

The first cups and/or the second cups may comprise wound dressing materials, and/or other materials as described herein.

Each of the first and second cups may be hermetically sealed and/or may comprise a sterile packaging.

According to a third aspect of the present invention, there is provided a method of treating a wounded breast, said method comprising fitting a bra, as described in the first aspect of this invention, to a subject to provide support to a breast. The subject may be a post operative subject and/or a subject who has suffered one or more injuries to a breast or breasts.

In use, the bra may be worn by an individual, such as a patient, to provide support to a breast (in for example a post-operative period) to reduce the risk of a wound infection, to promote wound healing and/or wound cosmesis.

The method may comprise removing and/or replacing the wound dressing material. The method may comprise replacing the wound dressing material by replacing a first or second component of the bra, a cup of the bra and/or by inserting or attaching a new wound dressing material into or onto a cup of the bra.

The method may comprise monitoring the healing progress via a window defined by the bra, such as a window exposing a wound or an area proximal and/or adjacent to a wound, the nipple and/or areole area.

The method may comprise fitting a modular bra to an individual. The method may comprise mounting a first component and a second component to a patient. The method may comprise releasably connecting the first and second components to one another to provide a bra. The first and second components may comprise cups of different sizes. For instance, the first and second components comprising different sized cups may be releasably connected to provide an asymmetric bra.

The method may comprise adjusting connectors at the front and back of an individual to allow for variations in intermammary space and/or ribcage diameter. The method may comprise adjusting, e.g. lengthening or shortening, shoulder straps of the bra. Adjustment of the bra may provide an improved fit of the bra to an individual, thus providing an improved support to the breasts. Improved support may facilitate the healing process. For example, by reducing excessive or unnecessary movement of the breast in a lateral or other direction.

As used herein a bra may mean a garment for providing support to one or both breasts. The bra may comprise a single cup to provide support to a breast, e.g. a treated breast. The bra may comprise a first and second cup to provide support to a first and second breast. At least one strap is configured to extend from a cup around the ribcage and back of an individual in order to mount the garment to the wearer. The bra may optionally comprise one, two or more shoulder straps to provide additional support to the wearer.

As used herein, a treated breast may mean a breast which has undergone a surgical procedure. An untreated breast may mean a breast which has not undergone a surgical procedure.

As used herein the term “post-operative” may refer to a period of time following a surgical procedure, such as immediately following a surgical procedure. In some cases, it may refer to the period of time it takes for any wounds or scars caused as a result of the surgical procedure to heal.

According to a fourth aspect of the invention, there is provided a garment for providing support to a wounded breast or a breast in a post-operative period substantially as described herein, with reference to the accompanying drawings.

In a fifth aspect, the invention provides a bra for providing support to a breast, said bra defining a window, wherein when in use the window defined by the bra exposes an area of the breast for observation. A bra according to the fifth aspect of this invention may comprise one or two cups. The bra may define a plurality of windows. The window or windows may be defined by parts of the bra cup. Where the bra comprises two cups, one or both cups may define one or more windows. Each cup may comprise, define or be divided into different zones. When in use (i.e. when being worn), the zone or zones of the bra (or cups thereof) may correspond to or sit above/adjacent to regions and/or areas of the breast affected by surgical procedures. Accordingly, the zones may correspond to, define or encompass known surgical incision/wound patterns. One or more of the zones may define a window permitting the wearer and/or a third party (for example a doctor) to observe and/or monitor an area of the breast and/or a wound. In this way, surgical wounds and/or tissue associated therewith may be monitored by, for example a doctor, throughout a post operative period.

For the avoidance of any doubt, it will be appreciated that any features described in detail for the first aspect of the invention may be equally applied to each of the second, third, fourth and fifth aspects of the invention.

One aspect of this invention provides a garment, for example a bra, for providing support to a breast, said bra comprising a cup or cups for supporting a or each breast, wherein one or both cups comprises a window as described above and a movable window covering (for example a flap) which is releasably fixable to the cup(s). The widow covering may be moveable between a first open position and a second closed position. In the closed position the covering/flap covers or obscures the window. The window may comprise (or consist essentially or consist of) an aperture defined by a, or the, cup(s) having disposed therein a transparent or opaque material. The window covering may define a pocket adapted to receive a re-symmetrising prosthesis. Moreover, in use and when the flap is releasably secured to the cup, a piece of wound dressing material (as described herein) may be purposefully trapped between the covering/flap and a window so as to facilitate wound healing. In this way, immediately after surgery, a patient who has lost some or all of their breast tissue can be re-symmetrised and/or reconstructed using prostheses which can be inserted into pockets defined by the moveable flaps/coverings. These prostheses can easily and temporarily be removed (by opening of a closure/flap) so as to expose a wound dressing which may be removed and replaced.

### BRIEF DESCRIPTION OF DRAWINGS

These and other aspects of the present invention will now be described, by way of example only, with reference to the accompanying drawings, in which:

FIG. 1a is a front view of an individual wearing a bra according to one embodiment of the invention;

FIG. 1b is an enlarged front view of an individual wearing a bra according to one embodiment of the invention;

FIG. 1c is a side view of an individual wearing a bra according to one embodiment of the invention;

FIG. 2 is a front view of a bra according to one embodiment of the invention;

FIG. 3a is an enlarged view of a front connector according to one embodiment of the invention;

FIG. 3b is an enlarged view of a front connector according to one embodiment of the invention;

FIG. 3c is an enlarged view of a front connector according to one embodiment of the invention;

FIG. 4a is a front view of a gauze pad configured for attachment or insertion to embodiments of the invention;

FIG. 4b is a profile view of the gauze pad shown in FIG. 4a;

FIG. 5a is a front view of pocketed cup according to one embodiment of the invention;

FIG. 5b shows the pocketed cup of FIG. 5a;

FIG. 5c shows a cross-sectional view of the cup of FIGS. 5a and 5b as taken along the line A-A;

FIG. 6a is an enlarged view of a prosthesis configured for attachment or insertion to embodiments of the invention;

FIG. 6b shows a profile view of a prosthesis according to one embodiment of the invention;

FIG. 6c shows a profile view of a prosthesis according to one embodiment of the invention;

FIG. 7a shows a front view of a support element according to one embodiment of the invention;

FIG. 7b shows a perspective view of the support element shown in FIG. 7a;

FIG. 8a shows a front view of a bra cup comprising a window according to an embodiment of the invention;

FIG. 8b shows a front view of a bra cup comprising a flapped window according to an embodiment of the invention; and

FIG. 8c shows a front view of a bra cup comprising flapped windows according to an embodiment of the invention.

FIG. 9: A—Internal projection showing opening (O), pocket (P) and fold (F) marking the start of the lateral component of the bra: B—External projection of the same showing pocket (P) and fold (F): C—Antero-posterior projection of bra on wearer showing position of opening (O), pocket (P) and fold (F) marking start of the lateral component of the bra.

FIG. 10: A—External projection showing laterally attached flap, pocket in flap (P) and the internal aspect of the bra cup (I). B—External projection showing superiorly attached flap and the under-surface of the flap (U). C—External projection showing inferiorly attached flap and highlighting the flap itself (F).

### DETAILED DESCRIPTION OF THE DRAWINGS

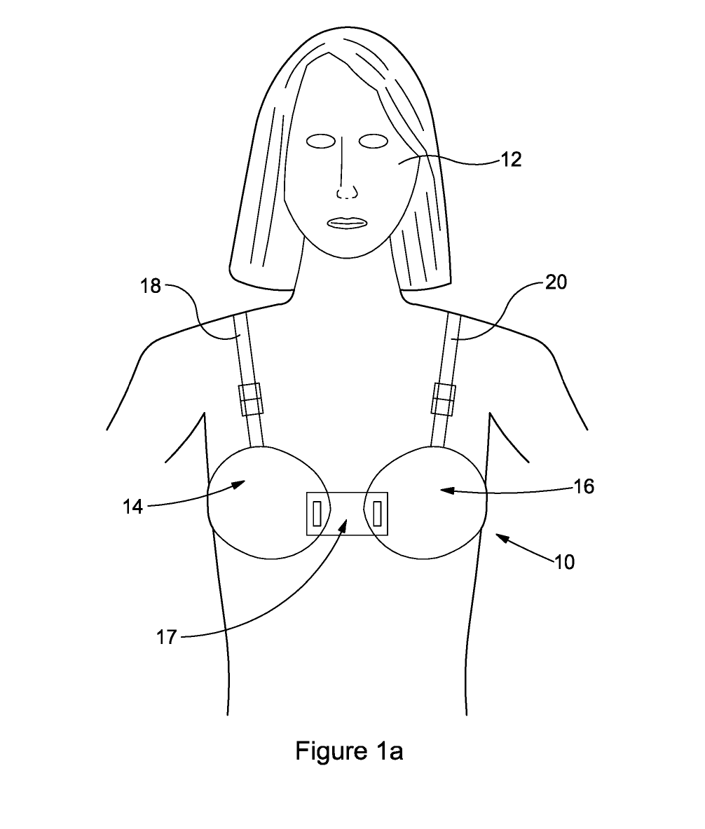

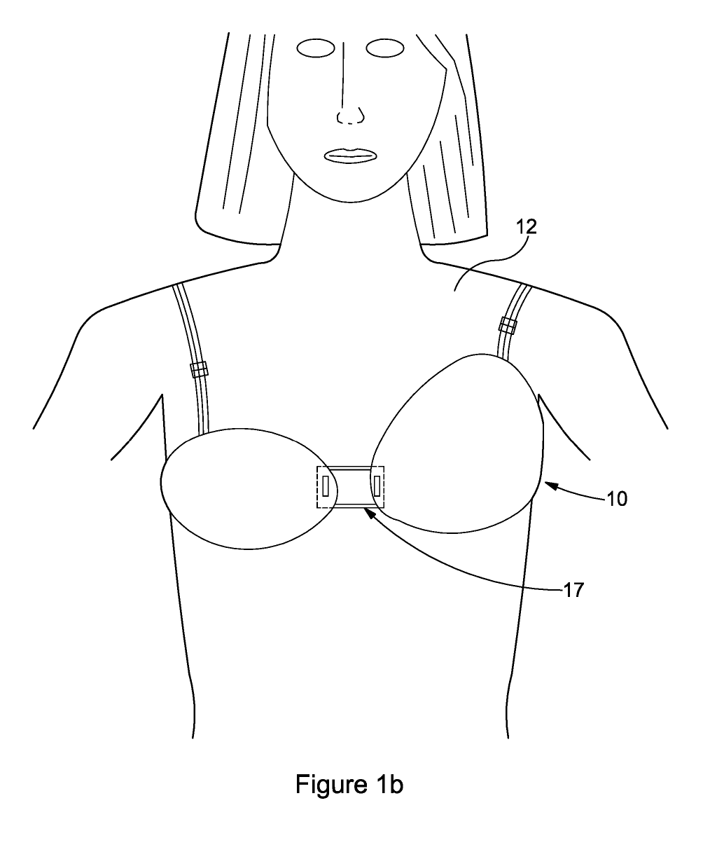

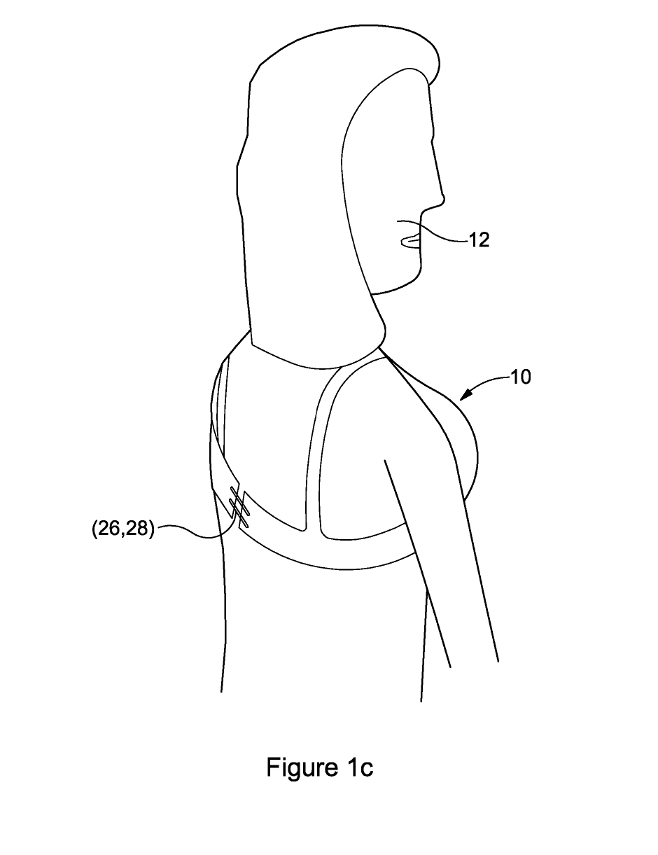

In use, a bra according to an embodiment of the invention is worn by a patient to provide support to a wounded breast or breasts. A breast wound may occur following trauma, for example, an accident or following a surgical procedure. FIGS. 1a to 1c show a bra (10) according to an embodiment of the invention being worn by an individual (12) to provide support to a breast. The bra (10) comprises a wound-dressing material and is mounted to its wearer using shoulder straps (18,20).

In FIGS. 1a to 1c, the breasts of the individual (12) are asymmetrical, which may, for example, be a result of surgery. In this embodiment, the cups (14, 16) of the bra (10) are of different sizes to provide support to each of the asymmetrical breasts. However, it will be appreciated that, in other embodiments, the cups (14, 16) of the bra may be of the same size. For example, to provide support to wounded symmetrical breasts or to resymmetrised breasts. FIGS. 1a to 1c also show connector element 17 located in the intermammary space which connects together cups 14 and 16.

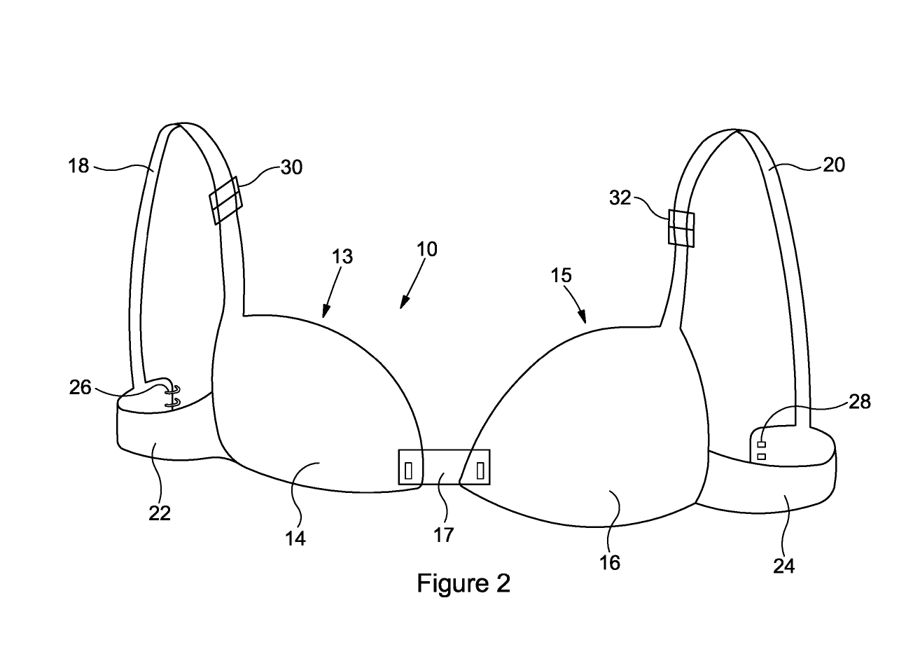

A bra (10) according to an embodiment of the invention is shown in more detail in FIG. 2. The bra (10) comprises a modular construction comprising a first (13) and second (15) component. The first component (13) comprises a first bra cup (14) and the second component (15) comprises a second bra cup (16). The first and second components are configured for attachment to each other so as to form a bra comprising two cups. The first and second components are releasably connectable anteriorly, e.g. between the breasts of an individual via a front connector (17). Additionally, the first and second components are releasably connectable posteriorly, e.g. at the back of an individual via a back connector comprising hook (26) and loop (28) connector elements.

The first and second cups (14,16) are each independently formed and adapted to at least partially envelope and/or support a breast. Straps (22,24) extend from the cups (14,16) and are configured to mount the bra to the wearer. These straps (22,24) are configured to be worn around an individual's back and ribcage. The bra (10) further comprises two shoulder straps (18,20) to provide additional support to the wearer. Although not shown on FIG. 2, the width of the shoulder straps (18,20) can be varied according to the cup size. For example, thinner shoulder straps may be used in conjunction with smaller cup sizes and wider shoulder straps may be used in conjunction with larger cup sizes. In addition, length adjustors (30, 32) are provided on each shoulder strap. The length adjustors (30,32) enable the shoulder straps to be independently lengthened or shortened after mounting the bra (10) onto an individual. Lengthening and/or shortening the straps (18, 20) in this way can provide improved support to a breast.

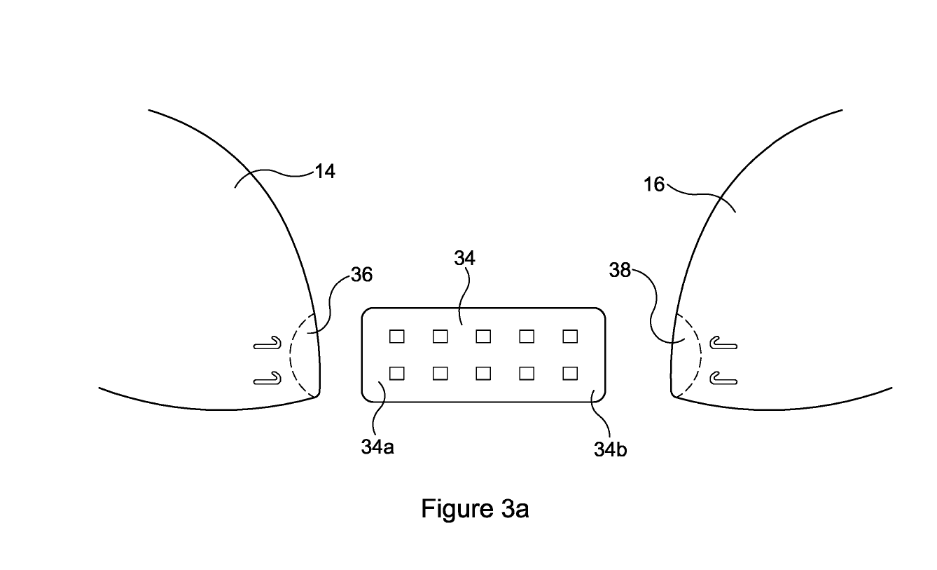

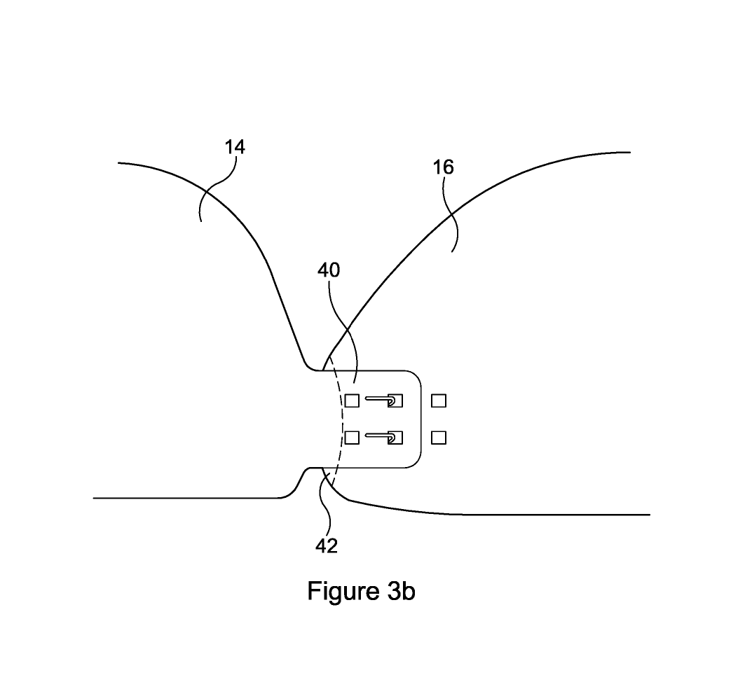

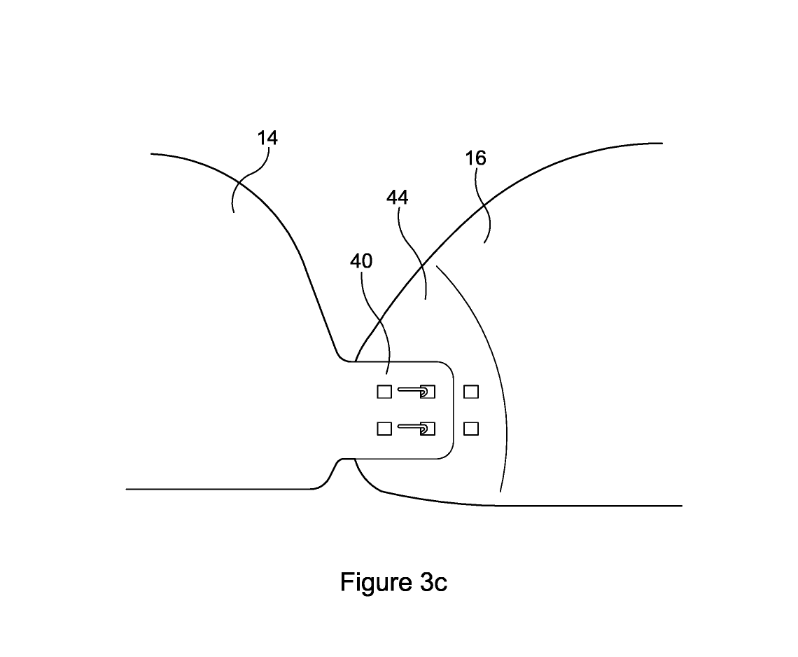

In the embodiment shown in FIG. 2, the straps (22,24) are releasably connectable at a plurality of discrete positions via a back connector (26,28). The two cups (14,16) are releasably connectable at the front of an individual via a front connector (17). The front connector (17) is releasably connectable at a plurality of discrete positions to accommodate any variation in the intermammary space between individuals. Various embodiments of the front connector are shown in FIGS. 3a to 3c and will be discussed in more detail in the following pages. The front (17) and back connectors (26,28) comprise connector elements, which are in the form of hooks (26) and loops (28) e.g. metallic or plastic hooks and loops. As will be appreciated, it is also possible to use other types of connector elements such as Velcro®, press studs, poppers or 3M Dual Lock® and the like.

The bra (10) comprises a wound dressing material. In some embodiments, the wound dressing material comprises an active agent. For example, the wound dressing material can comprise an antimicrobial agent, an antibiotic, an anti-bacterial agent, an anti-fungal agent and/or an anti-viral agent. In some embodiments, the wound dressing material comprises or further comprises an antimicrobial metal, an antibacterial metal and/or a material/metal having an oligodynamic effect e.g. the wound dressing material can comprise silver.

Additionally, or alternatively, the wound dressing material comprises a wicking and/or absorbant material. Materials of this type may assist the drainage of bodily fluids, such as pus, lymph or the like, from a wound.

In some embodiments, the wound dressing material comprises a breathable material. The breathable material can comprise a gauze-like material or a mesh-like material. In such embodiments, the wound dressing material allows circulation of air to the wound to promote wound healing.

As will be appreciated, any of the wound dressing materials described herein may be comprised in the bra (10) either alone or in combination. Representative examples of such wound dressing materials include, but are not limited to, antibacterial jersey fleece fabric (obtained from Lee Yaw Textile Co. Ltd), Torex® Makspec® (obtained from Toray), Infect-Protect (obtained from Raumedic®) and/or antimicrobial protected fabrics (obtained from Fab Industries).

The front connector shown in FIG. 3a comprises a central connector (34) one end of which (34a) is releasably connectable to a first cup (14) and the other end of which (34b) is releasably connectable to a second cup (16). In this embodiment, the first and second cups each independently define pockets (36, 38), formed between the material layers of the cups. The central connector (34) is insertable into the pockets (36, 38) and attachable to each of the first and second cups (14, 16) internally within the pockets (36, 38). The central connector (34) has outward facing connector elements that meet with their partners within the pockets.

The central connector (34) allows fine control of inter-mammary space and can be purchased separately if lost or damaged.

In an alternative configuration, shown in FIG. 3b, the front connector comprises a tab (40) extending from the first cup (14) and a pocket (42) comprised on the second cup (16). The tab (40) is insertable into the pocket (42) defined by the second cup (16). The tab (40) and pocket (42) comprise connector elements which are connectable after insertion of the tab (40) into the pocket (42). Although, as shown in FIG. 3c, the right cup comprises the tab and the left cup comprises the pocket, it will be appreciated that, alternatively, the right cup can comprise the pocket and the left cup comprise the tab. Either cup can comprise a tab (40) and/or a pocket (42).

In the connectors shown in FIGS. 3a and 3b, connector elements meet with partnering connector elements in an internal region of the bra, e.g. the pocket. Such a configuration avoids abrasion of the connector elements with the skin and/or an individual's clothing.

As will be appreciated, in alternative configurations, the pocket(s) can be omitted from the connectors shown in FIGS. 3a and 3b. For example, connector elements can meet with partnering connector elements in an external region of the bra. In such embodiments, the connector elements can meet with their partnering connector elements on the bra cups themselves. For example, in embodiments where the bra comprises a single layer of material.

Additionally, or alternatively, an area (44) comprising a connector element or in the vicinity of the connector element is reinforced as is shown in FIG. 3c. A reinforced area has properties of increased rigidity and/or strength. An area can be reinforced by the inclusion of rigid material into the bra, treatment of a material (e.g. by heat or chemical treatment or otherwise) comprised in the bra to increase its rigidity and/or strength, and/or by stitching over the area. For example, a rigid material can be placed between layers of material in the bra. Such reinforcement provides a secure anchorage area for the connector.

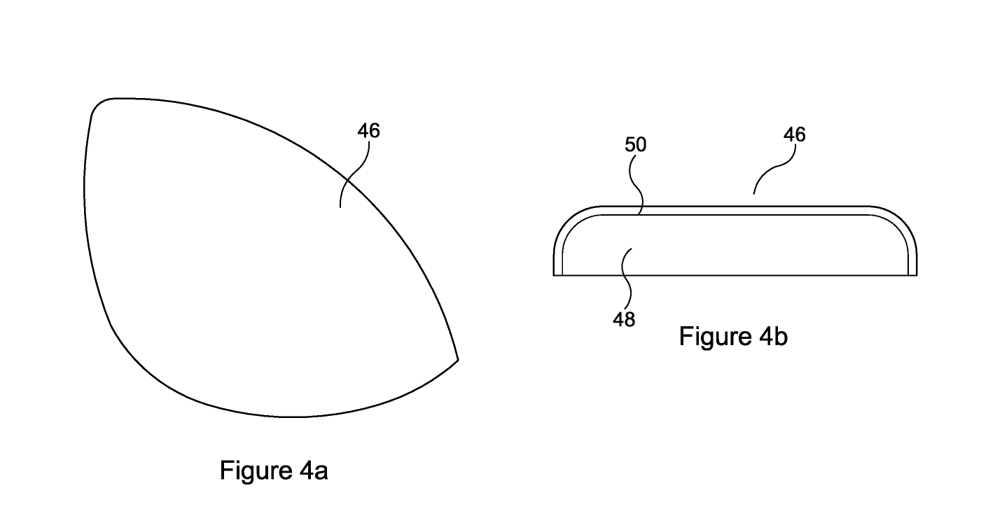

Shown in FIGS. 4a and 4b are examples of wound-dressing materials, in particular gauze pads, that are configured for insertion into and/or attachment to the bras described herein.

The gauze pad (46) shown in FIG. 4a comprises a layered structure. The inner layer (48) comprises a wound dressing material. The outer layer (50) comprises a non-wound dressing material, which in this embodiment comprises a liquid impermeable material. This prevents seepage of wound fluid onto a wearer's clothes and may assist in maintaining a dry environment for a wound. However, it will be appreciated that other non-wound dressing materials as described herein may additionally, or alternatively, be comprised in the outer layer (50), such as a non-medical material and/or an aesthetically pleasing material and, in some cases, the outer layer (50) of the gauze pad (46) may be absent. A pH indicator is also comprised in the outer layer (50). This provides a visual indication to a caregiver of when the gauze pad (46) requires changing or replacement.

In the gauze pad construction shown in FIG. 4a, the inner and outer layers (48, 50) are permanently attached by lamination. However, it will be appreciated that, in alternative configurations, the layers (48,50) can be attached via a different mechanism, or be releasably attached to one another.

As shown in FIG. 4b, the gauze pad (46) is configured to fit into a cup of the bra. The gauze pad (46) takes a similar form to the bra cup. When viewed in profile, the gauze pad (46) comprises a uniform thickness throughout its dimensions.

Although, as shown in FIGS. 4a and 4b, the wound dressing material comprises a similar shape to a bra cup, it will be appreciated that, in alternative configurations, each cup can comprise a plurality of wound dressing material pieces—each piece being independently removably attachable to the bra. For example, each cup can independently comprise, define or be divided into different zones, each zone independently comprising a removably attachable piece of wound dressing material, and the location of the zones corresponding to known surgical wound and/or scar patterns.

In use, wound dressing material can be attached to the zone or zones of the bra that correspond(s) to an expected wound and/or scar pattern on a patient before the bra is fitted to and/or worn by a patient. Post operation, the wound dressing material can be replaced as necessary—for example when saturated with fluid and/or as any active agent becomes ineffective.

Means of attachment of the wound dressing material, whether in the form of a gauze pad (46), a plurality material pieces or another form, to the bra (10) include an adhesive or a stitched seam or a fastening mechanism, such as a hook-and-loop type mechanism e.g. Velcro®, buttons, press studs, zip fasteners, or the like. Alternatively the wound dressing material may be placed into a pocket defined by the bra.

In certain embodiments, a cup of the bra (10) is unpocketed and constructed from a single layer of antibacterial, wicking material. In some cases, this material is slightly adherent to allow attachment of a gauze pad (46) or plurality of material pieces for collection of wound fluid.

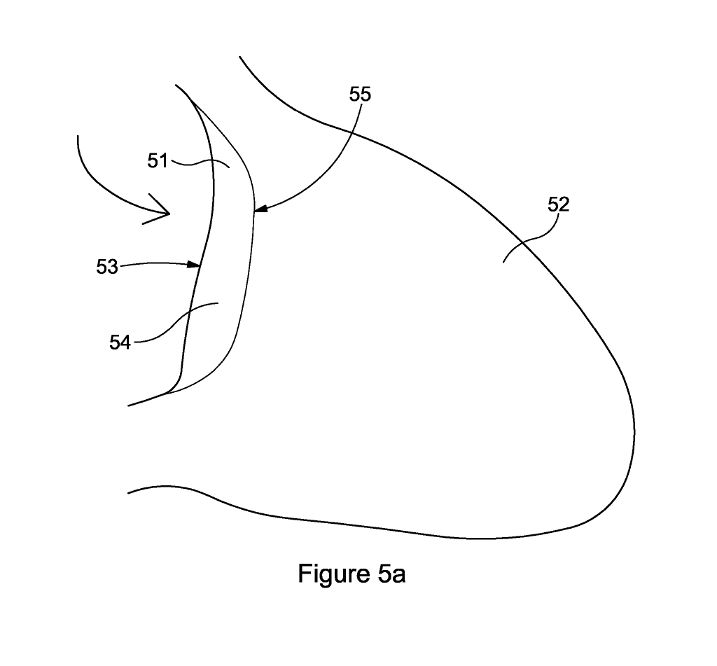

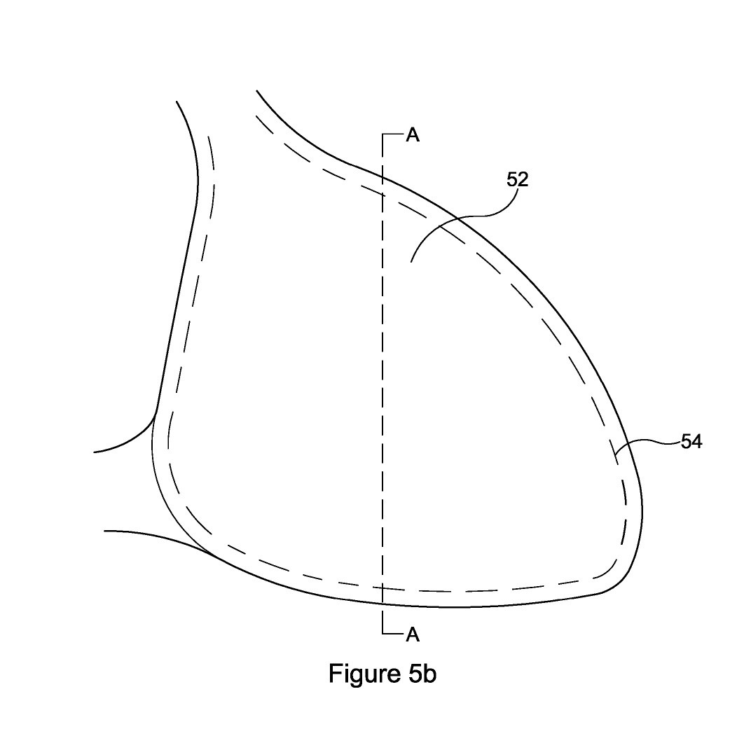

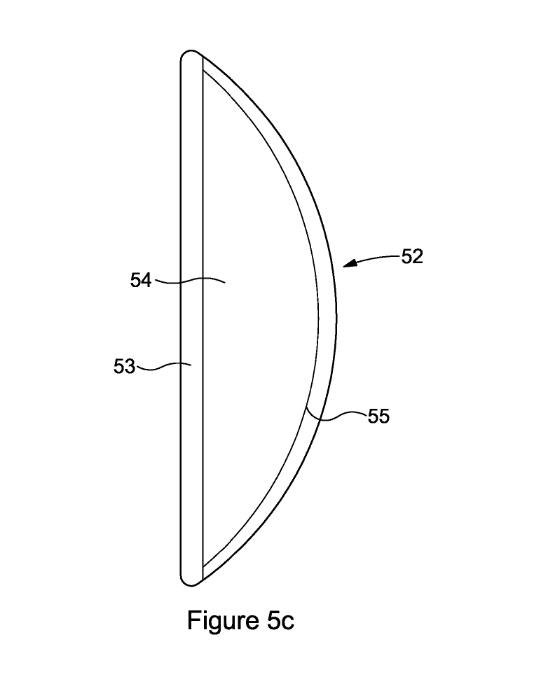

In the example configuration shown in FIGS. 5a to 5c, the bra (10) comprises a pocket (54) defined by the cup (52). The outline of the pocket (54) dimensions is shown by the dotted line on FIG. 5b and a cross-sectional view of the pocket taken through plane A-A on FIG. 5b is shown in FIG. 5c. The pocket (54) is defined by the inner (53) and outer (55) layers of the bra cup (52). The pocket (54) defines an opening (51) into which a wound dressing material, such as a gauze pad, can be inserted. Additionally or alternatively, a prosthesis (56) can be inserted into the pocket (54).

As shown in FIG. 5a, the opening (51) of the pocket (54) is located in a lateral position. This allows access to the pocket (54) from the side of the cup (52). As will be appreciated, in other embodiments, the opening can be located at any position on the bra cup. For example, the opening can be located centrally on the bra cup. This would allow access to the pocket (54) from the front of the cup.

In some embodiments, the inner layer (53) comprises a foam-padded cotton material and the outer layer (55) comprises an elastic and/or smooth material to allow insertion of a prosthesis (56) e.g. an ultra-stretchy and/or smooth Lycra®-based material.

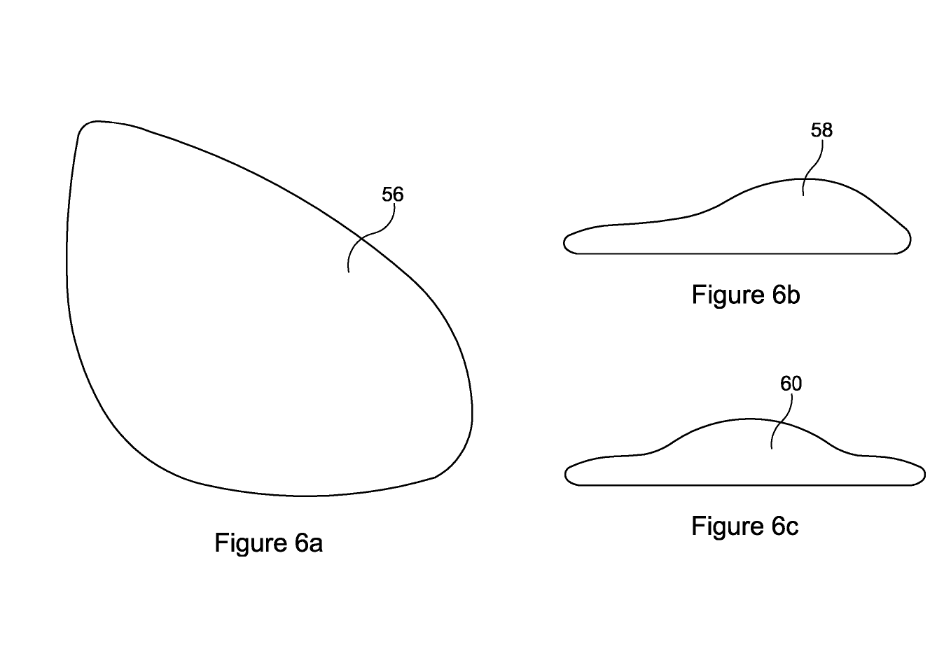

As shown in the examples of FIGS. 6a to 6c, a variety of shapes of prosthesis (56) can be inserted into pockets of the bras. In each case, the prosthesis (56) is configured to provide a flattering profile to the wearer. For example, in some cases the prosthesis is configured to restore a pre-surgery profile to a breast and/or to provide a symmetrised breast profile to an individual. The example prosthesis shown in FIG. 6b comprises a teardrop-like surface profile (58). The teardrop-like surface profile adds volume to either the upper or lower breast pole as required. The example prosthesis shown in FIG. 6c comprises a rounded surface profile (60). The rounded surface profile adds volume to a breast centrally. In each case, the prostheses (58, 60) comprise a tapered profile, being thinner in the edge areas, to allow a more flattering profile to the wearer when wearing thin undergarments. The prostheses may comprise gel, silicon, water and/or saline.

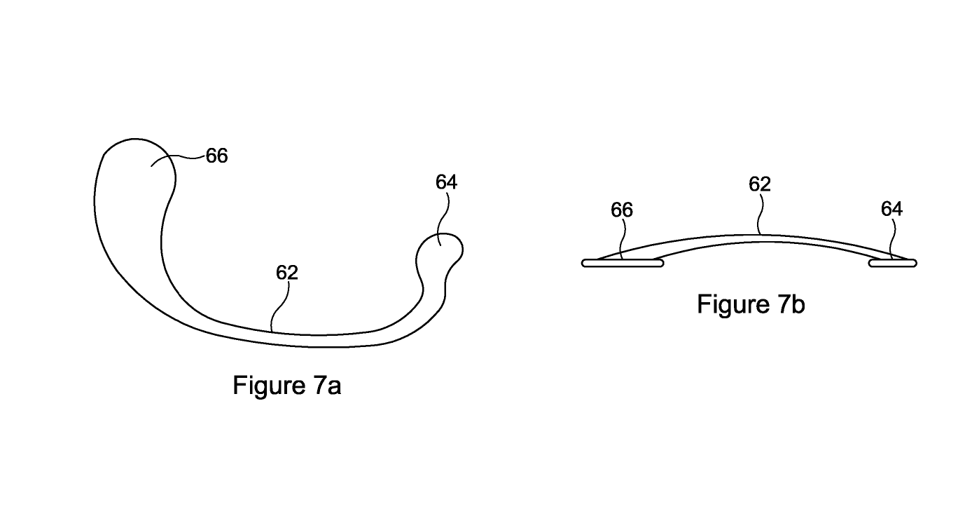

In some embodiments of the bra (10), one or both of the cups comprise a support element configured to provide support to the underside of a breast. An example of such a support element (62) is shown in FIGS. 7a and 7b. The support element (62) comprises a substantially planar arcuate shape terminating in enlarged head portions (64, 66) at each opposing end of the element (62). A lateral head portion (66) is larger than a medial head portion (64). Such a construction provides an improved amount of lateral support to the breast, whilst reducing congestion in an intermammary space. The head portions (64, 66) are rounded and/or flattened to reduce the risk of irritation or abrasion on the breast area. The support element (62) comprises a rigid or semi-rigid material, such as a plastics material or a metallic material.

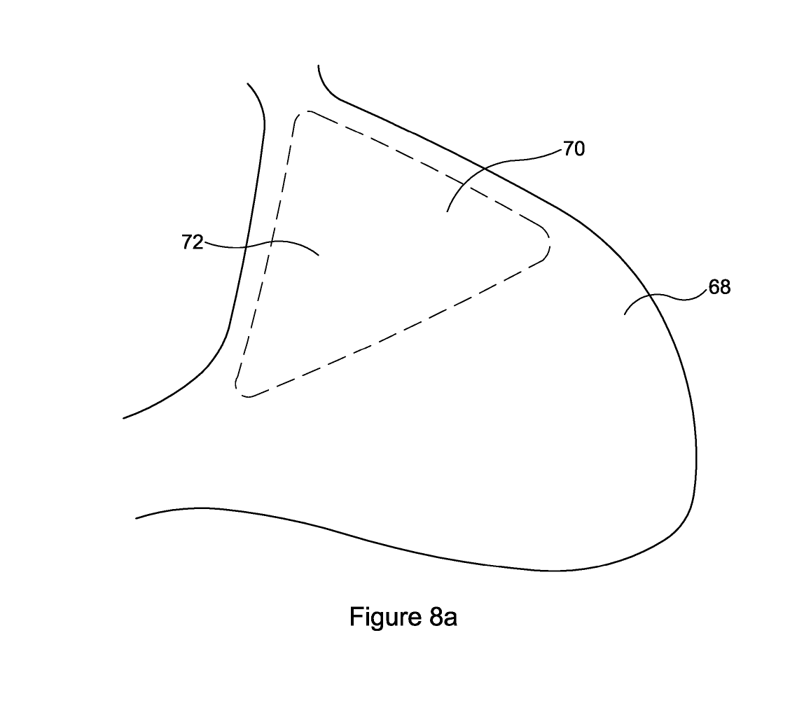

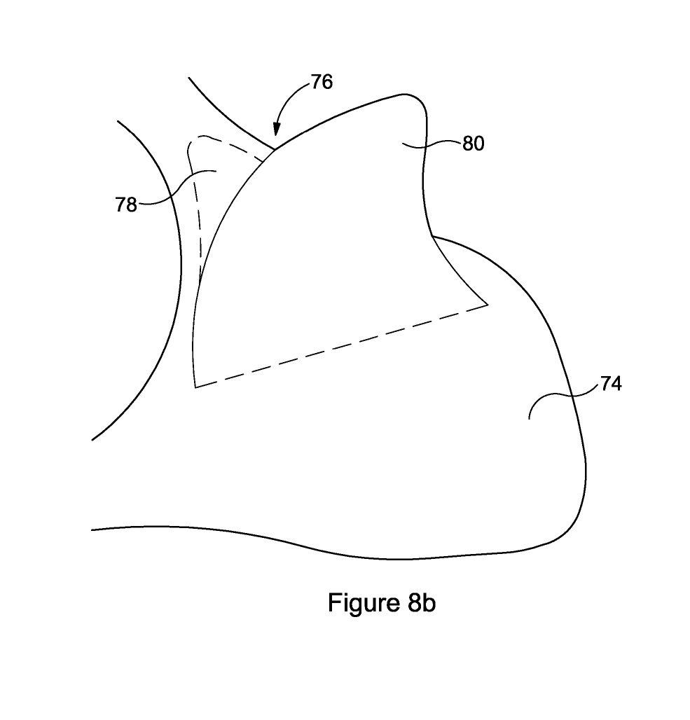

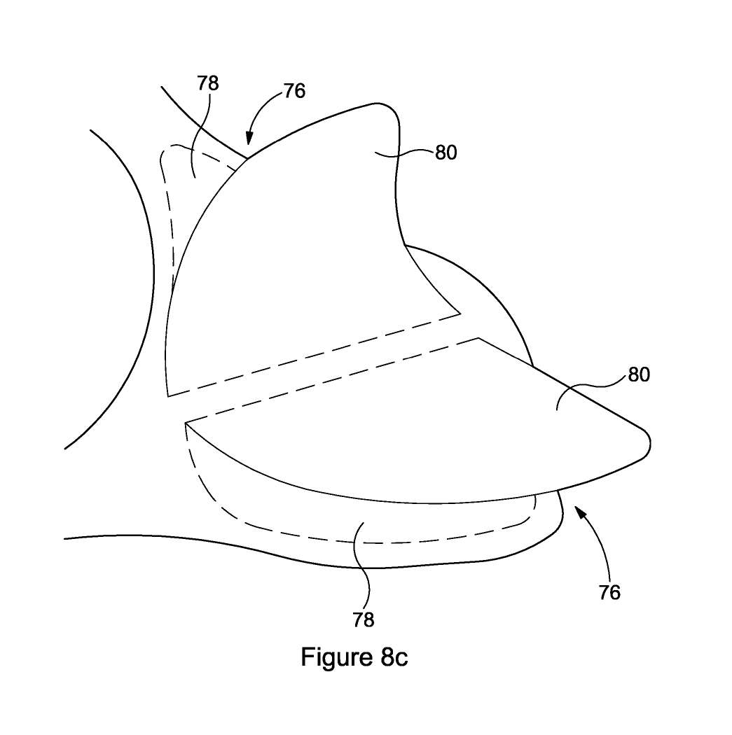

In the embodiment shown in FIG. 8a, the bra (10) comprises a cup (68) which defines a window (70). The window (70) enables a user and/or a third party to monitor the healing progress of a wounded breast by observation. The window (70) exposes an area of the breast, for example an area of the breast proximal to and/or associated with, a wound. The window (70) allows a third party to observe at least part of a wound directly and/or an area of the breast proximal to and/or associated with, a wound. Although not shown in FIG. 8a, in some embodiments, the window (70) exposes and/or allows a user and/or third party to observe, all or part of the wound dressing material.

Although only one window is shown on the cup (68) of FIG. 8a, it will be understood that, in other embodiments, the bra defines a plurality of windows, each exposing all or part of a breast wound, area of the breast associated with or proximal to, a wound and/or wound dressing material of the bra.

In the embodiment shown in FIG. 8a, the window (70) defined by the bra is configured for location over an upper area of the breast. However, it will be appreciated that in other embodiments, the window (70) can be configured for location in alternate positions dependent on the location of the wound and the tissue in proximity to it. For instance, the window (70) can be configured for location over a wound, a nipple and/or areole area or proximal to any of these sites, to allow a medical professional, such as a nurse or doctor, to monitor the healing progress of a wounded breast, e.g. to monitor tissue health, viability and healing, in a post-operative period. Such an observation window allows a medical professional to monitor the healing progress without the need for removing either the bra and/or any wound dressing material thereof. As will also be appreciated, in some embodiments, a bra of this invention can define one or more windows in pre-determined positions, which positions are intended to correspond to areas of the breast affected by surgical procedures.

The window (70) comprises an aperture (72) defined by the bra. In some embodiments, the window (70) comprises a transparent, opaque and/or mesh-like material disposed within the aperture (72) defined by the bra.

As shown in FIGS. 8b, in some embodiments, the window (76) comprises a flap (80) configured to cover the aperture (78). The flap (80) is moveable between a closed position and an open position. In the closed position, the flap (80) covers the aperture (78). In an open position, the flap (80) is positioned away from the aperture (78). In some embodiments, the flap (80) comprises an opaque material and observation through the window (76) is possible when the flap (80) is both in the closed and open positions. In some embodiments, the flap (80) comprises a liquid impermeable material. In such configurations, when in a closed position, the flap (80) protects a wound, breast tissue in the vicinity of, and/or associated with, a wound or wound dressing material of a bra from liquid ingress and assists in maintaining a dry environment. In some embodiments, a bra comprises a plurality of flapped windows as is shown in FIG. 8c.

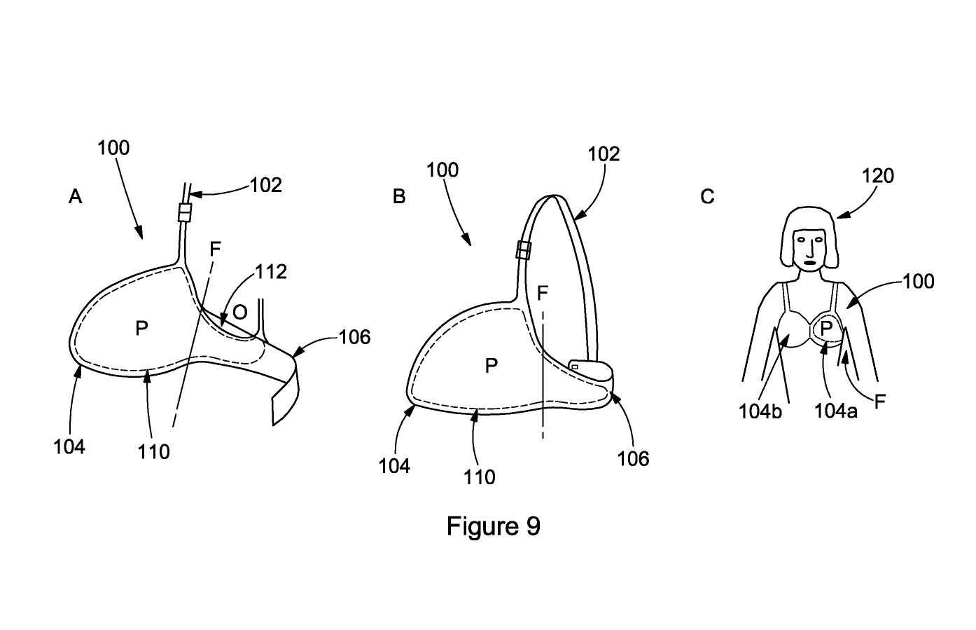

FIG. 9 shows a bra 100 according to a further embodiment of this invention. FIGS. 9A and 9B show only the left cup 104 portion of the bra 100, which portion comprises a cup 104 and shoulder straps (102) for mounting the bra portion on a wearer. FIGS. 9A and B also show the lateral bra portion 106 which in use wraps around the side of the wearer's body. In FIG. 9B, the start of the lateral portion 106 is marked by line “F”. Bra 100 defines a pocket 110 and an opening 112 thereto. Pocket 110 spans substantially the entirety of the cup 104 and is adapted to receive (via opening 112) a prosthesis and/or wound dressing material. FIG. 1B shows that in use and when correctly worn, opening 112 becomes closed preventing access to pocket 110.

FIG. 9C shows a complete bra comprising two cups 104a and 104b being worn. In this figure the location of packet 110 relative to the wearer 120 is shown. It should be noted that while the fissure only shows that cup 104a comprises a pocket 110, cub 104b may comprise a similar or different pocket. Although it is not clearly shown in FIG. 9C, opening 112 is sited within the lateral component 106 of the bra (best shown in FIGS. 9A and B); as a consequence, access to the pocket 110 would be limited by temporary closure of the opening 112 resulting from the compression generated on the lateral component 106 of the bra 100 as it is worn. Access to pocket 110 via the opening 112 will be available when the bra 100 is removed and/or there is no compression on the lateral components 106 of the bra 100.

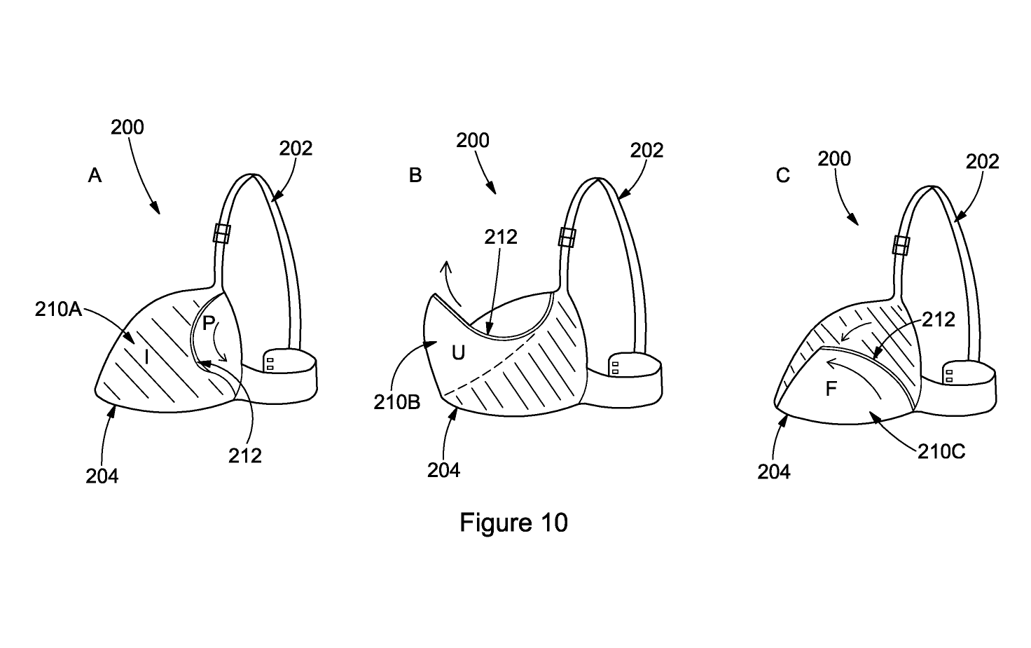

FIGS. 10A-C, show a bra 200 according to a further embodiment of this invention. FIGS. 10A and 10B show only the left cup 204 portion of the bra 200, which portion comprises a cup 204 and shoulder straps (202) for mounting the bra portion on a wearer. FIGS. 10A and B also show the lateral bra portion 206 which in use wraps around the side of the wearer's body. Bra 200 includes a movable flap of material 210A, 210B and 210C. Flap 210A/B/C may be lowered or raised and is releasably attachable to the bra cup 204 by fixings not shown (such fixings may include loop and hook fixings and the like). Flap 210A/B/C is attached to the bra by at least one axis at all times. FIG. 10A, shows a lateral flap 210A attachment; FIG. 10B a superior flap 210B attachment and FIG. 10C; an inferior flap 210C attachment. The flap components are separate to any components of the bra cup 204 which remains in place and integral at all times during flap 210A/B/C operation. As shown in FIG. 10A, flap 210A comprises a number of layers and defines a pocket 212 and an opening thereto 214. Pocket 212 may be adapted to receive a disposable dressing pad or a re-symmetrising prosthesis. The under or inner surface (for example surface U shown in FIG. 10B) of any of the flaps 210A/B/C flap may be made from conventional material or a material that is water-impermeable but breathable. Flap 210A/B/C may be realisably attached to the bra 200 in such a way that, in use, the flap 210A/B/C can be used to trap a piece of wound dressing material and/or a prosthesis (or some other device) therebetween (i.e. between the flap 210 A/B/C and the cup 204). This arrangement may allow for the interposition of a disposable wound dressing pad between the cup 204 and the flap 210A/B/C and, for example, the insertion of a prosthesis into a pocket 212 of the flap 210A/B/C.

1. A garment for providing support to a breast, said garment comprising a wound dressing material, wherein the garment comprises at least one cup, which cup is formed and adapted to at least partially envelope and/or support a breast.

2. The garment of claim 1, wherein the garment is a bra comprising two cups, each cup formed and adapted to at least partially envelope and/or support a breast.

3. The garment of claim 2, wherein the garment is an asymmetric bra.

4. The garment of claim 1 defining one or more windows, wherein when in use each window of the garment independently exposes a wound, an area of the breast and/or wound dressing material for observation.

5. The garment of claim 4, wherein the location of the window(s) corresponds to known surgical incision/wound patterns.

6. The garment of claim 4, wherein one or more of the window(s) comprises an aperture defined by the garment and optionally wherein the window comprises a transparent, opaque and/or mesh-like material disposed within the aperture defined by the garment.

7. The garment of claim 6, wherein the aperture defined by one or more of the window(s) comprises wound dressing material.

8. The garment of claim 7, wherein the wound dressing material comprises an active agent, optionally an agent having an antimicrobial/antibacterial effect and/or a material having an oligodynamic effect, such as silver.

9. The garment of claim 1, wherein the garment comprises, defines or is divided into different zones.

10. The garment according to claim 9, wherein each zone independently comprises a wound dressing material and/or a removably attachable piece of wound dressing material.

11. The garment according to claim 9, wherein the location of the zones corresponds to known surgical incision/wound patterns.

12. The garment of claim 1, wherein the garment further comprises a movable flap or cover.

13. The garment of claim 12, wherein the flap or cover is moveable between a closed position and an open position.

14. The garment of claim 13, wherein the flap or cover is located on the cup portion of the garment.

15. The garment of claim 12, wherein the flap or cover is configured or adapted to releasably cover a window, aperture or zone defined by the garment.

16. The garment of claim 12, wherein the flap or cover defines a pocket for releasably receiving a piece of wound dressing material and/or a prosthesis.

17. The garment of claim 16, wherein the prosthesis is a re-symmetrising or reconstructing prosthesis.

18. The garment of claim 1, wherein the at least one cup thereof defines a pocket, optionally wherein the pocket defines an opening into which wound dressing material may be inserted.

19. The garment according to claim 18, wherein the pocket defines an opening into which a prosthesis may inserted, the prosthesis being configured to provide a re-symmetrised and/or reconstructed breast profile to an individual, optionally wherein the prosthesis comprises gel, silicon, water and/or saline.

20. The garment of claim 1 wherein the wound dressing material comprises an active agent, optionally a material having an oligodynamic effect, such as silver.

21. The garment of claim 1, wherein the bra has a modular construction and comprises at least a first and second component configured for attachment to each other so as to form a bra comprising two cups.

22. A medical kit comprising a plurality of garments according to claim 1, wherein each garment comprises one bra cup configured for connection to another bra cup.

23. A medical kit according to claim 22, wherein the kit comprises a plurality of first bra cups and second bra cups, wherein the first and second bra cups are configured for interchangeable attachment to one another to provide a bra comprising two cups, the bra comprising a wound dressing material.
